(19) United States
(12) Patent Application Publication
(10) Pub. No.: US 2023/0386620 A1
(43) Pub. Date: Nov. 30, 2023

(54) SYSTEMS AND METHODS FOR DETECTION OF POTENTIAL MEDICAL CONDITIONS

(71) Applicant: Genome Medical, Inc., South San Francisco, CA (US)

(72) Inventors: Kiley Jo Johnson, Chatfield, MN (US); Mark Edward Sylvester, Wayzata, MN (US); Ashley VanDenBoom Daley, Inver Grove Heights, MN (US); Andi Hila, Columbus, OH (US); Durand R. Van Arnem, Apple Valley, MN (US); Colleen Anne Caleshu, Bend, OR (US); Kaylene Ready, Sugar Land, TX (US); Jill Davies, Toronto (CA); Cary Armstrong, Centennial, CO (US); Callan Delaney Russell, McDonough, GA (US)

(21) Appl. No.: 18/322,806

(22) Filed: May 24, 2023

Related U.S. Application Data
(60) Provisional application No. 63/346,797, filed on May 27, 2022.

(51) Int. Cl. G16H 10/20; G16H 50/20

(52) U.S. Cl. CPC G16H 10/20; G16H 50/20

(57) ABSTRACT

Approaches for generating recommendations for a user related to genetic counseling are provided. A user can be provided with one or more first questions that may be relevant to one or more medical questions. Based at least in part upon one or more answers to the first questions, one or more second questions can be determined and provided to the user. The user can be classified into one or more categories based at least in part upon answers provided to the first questions and the second questions. A recommendation for the user can be generated based at least in part upon the classification of the user.

### CROSS-REFERENCE TO RELATED APPLICATION

This application claims priority to, and the benefit, of U.S. Provisional Patent Application No. 63/346,797, filed May 27, 2022, titled “SYSTEMS AND METHODS FOR DETECTION OF POTENTIAL MEDICAL CONDITIONS,” the full disclosure of which is hereby incorporated in its entirety for all purposes.

### BACKGROUND

Patients may wait to seek out genetic testing for underlying medical conditions, often because they do not even realize they need genetic testing. For example, 90% of patients who would otherwise meet the guidelines for hereditary cancer testing are not appropriately identified, leaving a significant pool of the human population who are at risk for cancer. Early detection is often key in cancer treatment. However, the time required for speaking to a counselor about early screening may discourage a patient from speaking to a counselor to begin with. Additionally, online questionnaires may be overly cumbersome for a patient to fill out, as there may be hundreds of questions that could be considered pertinent to a diagnosis. Therefore, there is a need for a way to detect cancer early, in a quick and efficient manner.

### BRIEF DESCRIPTION OF THE DRAWINGS

Various embodiments in accordance with the present disclosure will be described with reference to the drawings, in which:

FIG. 1A illustrates an example system that can be utilized in accordance with the various embodiments.

FIGS. 1B and 1C illustrate example interfaces that can be used to implement one or more aspects of the various embodiments.

FIG. 2 illustrates an example decision tree that can be used in accordance with the various embodiments.

FIG. 3 illustrates an example method that can be used in accordance with the various embodiments.

FIG. 4 illustrates an example method that can be used in accordance with the various embodiments.

FIG. 5 illustrates an example environment for implementing aspects of the various embodiments of the present disclosure.

FIG. 6 illustrates an example provider network (or “service provider system”) environment according to some embodiments.

FIG. 7 is a block diagram of an example provider network that provides a storage service and hardware virtualization service to customers, according to some embodiments.

FIG. 8 illustrates a system that implements a portion or all of the techniques described herein, according to some embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

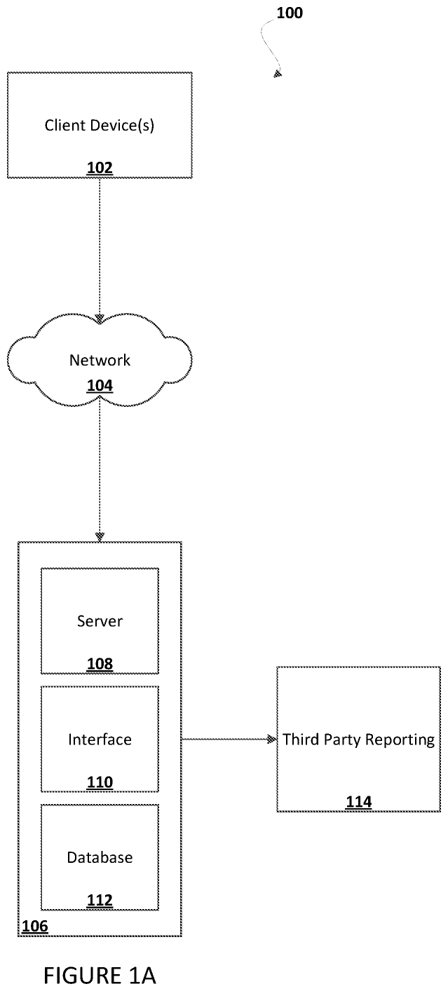

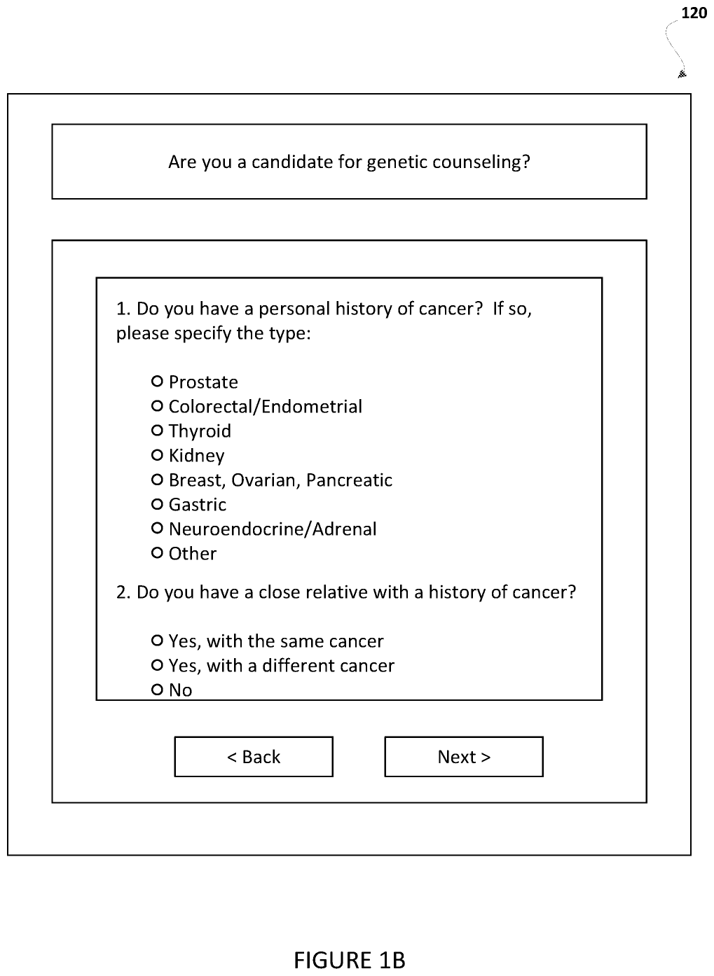

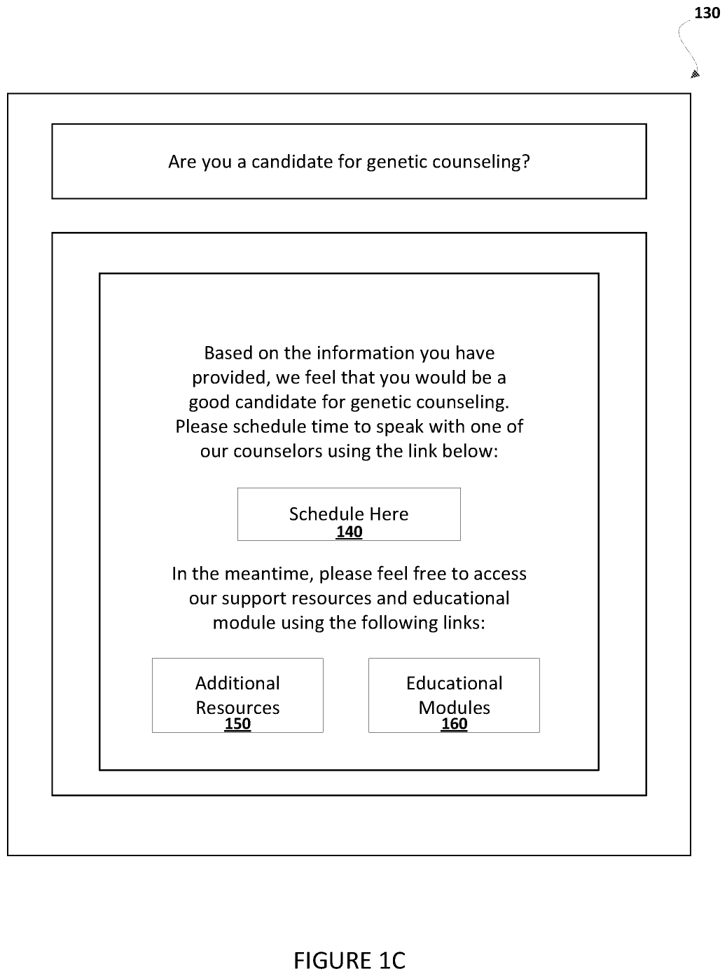

Approaches for detecting potential medical conditions are presented herein. FIG. 1A illustrates an example system 100 that can be utilized in accordance with the various embodiments. The system includes one or more electronic client devices 102, which can include any appropriate device operable to send and receive requests, questions, messages or other information over an appropriate network 104 and convey information back to a user of the device. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the system 100 includes a server environment 106 including a server 108 for receiving requests and serving content in response thereto, although for other networks, an alternative device serving a similar purpose could be used, as would be apparent to one of ordinary skill in the art.

The server environment 106 includes at least the database 112 and the interface 110 for storing content and information and serving content to the client device(s) and third party reporting 114. It should be understood that there can be several application servers, layers or other elements, processes or components, which may be chained or otherwise configured, which can interact to perform tasks such as obtaining data from an appropriate data store. As used herein, the term database refers to any device or combination of devices capable of storing, accessing and retrieving data, which may include any combination and number of data servers, data storage devices and data storage media, in any standard, distributed or clustered environment. The server 108 can include any appropriate hardware and software for integrating with the database 112 as needed to execute aspects of one or more applications for the client device and handling a majority of the data access and business logic for an application. The server 108 provides access control services in cooperation with the database 112 and is able to generate content such as text, graphics, audio and/or video to be transferred to the user, which may be served to the user via the interface 110 in the form of HTML, XML or another appropriate structured language in this example.

The system 100 in one embodiment is part of a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 1A. Thus, the depiction of the system 100 in FIG. 1A should be taken as being illustrative in nature and not limiting to the scope of the disclosure.

FIGS. 1B and 1C illustrate example interfaces 120, 130 that can be used to implement one or more aspects of the various embodiments through the system 100 of FIG. 1A. In at least one embodiment, a risk assessment module for early genetic screening is provided. A risk assessment module according to an example embodiment may utilize a series of questions for a patient to answer, or other mechanisms for gathering data for a patient, and may provide output such as a recommendation or summary as to whether the patient should seek genetic counseling, information regarding risk facts, classifications, groupings, educational information, and/or the like. Additionally, a clinician may be provided with the user's information and/or results, as may include various questionnaire answers and status updates. Questionnaire and results may be provided in physical or digital form. If in digital form, the questionnaire and results may be provided via a computer, such as a laptop, smartphone, or tablet device.

An example questionnaire may include information that a specialist or counselor may ask, in addition to questions based on nationally-recognized guidelines and other questions which may be pertinent to early detection of a medical issue. Less pertinent or repetitive questions may be filtered out, leaving behind a specific subset of questions which may improve the likelihood of an early but accurate detection of a patient that would likely qualify for genetic testing. The overall time required for a user to fill out the questionnaire may be reduced to only a few minutes, instead of several minutes to an hour or more, due to the structured nature of the questionnaire. Moreover, the questionnaire, based on input responses provided by the user, may add or remove downstream questions, thereby streamlining the approach for the user. As previously mentioned, a user may spend significant amounts of time if they were to answer every single question in nationally-recognized guidelines or if the user were to see a specialist or counselor for early detection of a medical issue. Embodiments of the present disclosure overcome this problem by using one or more initial questions to identify a workflow to address one or more possible conditions that may be recommended for monitoring and/or testing.

According to an example embodiment, a list of risk factors or criteria identified for a type of medical condition may be provided to a user. For example, one or more risk factors or criteria for a specific type of cancer may be determined. Of those risk factors, a subset of risk factors determined to be most pertinent to the medical condition may be selected and/or identified. The subset of risk factors may be presented to a patient or user, such as in the form of a questionnaire. The subset of risk factors may include those factors which may be commonly attributed to an underdiagnosis of the targeted condition. In an example, risk factors may correspond to one or more criteria, such as “personal history of prostate cancer at any age” and “close relative with ‘X’ cancer”, where “X” denotes a specific type of cancer. A “close relative” according to this example may refer to a relative on the same side of the family who is a first, second, or third degree relative of the patient. A combination of criteria may be considered when determining whether to refer a patient to genetic counseling, such as age and history of cancer in combination with a known family history of cancer. In another embodiment, only a single criterion may need to be met in order to determine with sufficient confidence that the patient should be referred for genetic counseling. For example, a user having a known familial mutation or a relative with a known familial mutation regardless of personal or family history of cancer may always be referred for genetic counseling. The use of these criteria may improve the accuracy of a clinical recommendation, and may enable a user to seek genetic counseling earlier than they otherwise would have. Moreover, if a confidence level is high enough with fewer questions, resources may be saved for both the user (e.g., time) and the system itself (e.g., processing, bandwidth, etc.). Additionally, given the convenience of being able to take a shortened questionnaire at home, a user does not have to spend time visiting a physical site to determine whether to seek genetic counseling.

In some cases, a given risk factor or criterion may require specific sub-rules, such as “a patient's personal history of (a specified type of) cancer does not meet this criterion” when the questionnaire pertains to a different type of cancer. Other sub-rules may include “must be separate close relatives with cancer on the same side of the family, not one relative with multiple cancer diagnoses,” among other such relationship mappings. The criteria may be associated with a frequency score indicating how frequent a particular combination of criteria arises in a clinical visit. For example, patients may frequently be referred for genetic counseling if they have both a personal history of prostate cancer and the prostate cancer is metastatic. This information may be updated as new patient data is collected, and the information may be fed into a machine learning-based training system as training data to improve the prediction of potential risk for patients. Furthermore, as data is collected, new correlations and rules may also be developed, which may be mapped by clinicians or by one or more additional machine learning systems.

A classifier may be utilized to determine whether a patient is considered to be high-risk. The classifier may be a machine learning-based classifier. For each question answered with an affirmative answer, as an example, a score may be generated. Additionally, scores may also be generated when the user selects an answer from a list of possible answers, where different answers within the list have different scores/weights. The scores for the questions may be aggregated and compared to one or more threshold values. The classifier may determine which recommendation to provide to the user depending on how the aggregated score compares to the threshold values. For example, if the aggregated score exceeds a threshold value, a recommendation to get tested for a genetic condition may be provided to a user. Additionally, a recommendation to seek counseling for a medical condition may be provided. In an example, an indication about whether a user is high risk may be provided. If a score falls between two threshold values, an indication about a user being at risk, but not necessarily at high risk, may be provided. If a score falls below a determined threshold value, an indication that the user is low-risk may be provided, or if the risk is sufficiently low, then no output may be provided regarding risk.

In accordance with an example embodiment, the classifier may also consider secondary information, such as whether the patient has an insurance policy that would cover a genetic counseling session. With the secondary information, the classifier may consider particular nuances which could affect whether a patient should be recommended for genetic counseling. Other secondary information may be considered, including information indirectly related to a particular genetic condition.

In another embodiment, one or more weightings may be assigned for individual factors. For example, if one factor tips more heavily than others in favor of referring a user for genetic testing, that factor can be assigned a higher weighting or multiplier for the individual score. In this way, the aggregated score may be a more reliable score when used to determine whether to recommend genetic testing/counseling.

As shown in the example interface 130 of FIG. 1C, if a patient is determined to be high risk based on the questionnaire (or considered a good candidate for testing regardless of risk in embodiments where risk levels are not evaluated), they may be provided the option of scheduling an appointment 140 with a specialist or genetic counselor to discuss their results. Additionally or alternatively, a patient may be provided with a copy of their results in a comprehensive report. Such a report may include individual responses by the patient, along with a summary indicating whether a user should seek genetic testing for a potential medical condition. The results may be displayed on a screen after the initial questionnaire has been filled out, or the results may be sent electronically to the patient. Furthermore, the patient may receive additional items regarding the report, such as whether to provide the information to their primary care physician and/or providing consent to use the information for additional purposes, such as updating training data.

The criteria used in the risk questionnaire may be individually validated against clinical results received for a user. For example, an output from the risk analysis system may be compared to a recommendation provided by a clinician to determine whether the risk analysis system erroneously triggered a recommendation that it should not have. This information may be utilized to further train and fine-tune the system, to prevent false positives in future cases. This training system may significantly reduce the amount of time a patient may spend in seeking genetic counseling. In at least one embodiment, this information may be anonymized prior to processing as training data to maintain privacy for user information. Additionally, users may be asked to “opt-in” to using their information for further training or evaluation purposes, and as a result, a provider may seek and receive content prior to using the information for further training purposes.

Additionally, the criteria may be assessed for undercall, overcall, or missed results. For example, a personal history of prostate cancer may have resulted in a miss because an initial questionnaire may have only asked whether the family history of prostate cancer was metastatic, without other details such as the stage or other pathology details, because the patient is not expected to know these details about their family members' diagnosis. As another example, a user may be asked about their personal history of prostate cancer and whether they have a brother or father with prostate cancer who is over 60 years old. This may result in an overcall, but this is because a patient will not likely know the more intimate details about the cancer itself. In such cases, the patient may be referred to a clinician to discuss those details, but the user may be presented with an information request so that the user may fill out the lesser-known details prior to their appointment. Additionally, or alternatively, the user may be provided with credentials to securely log-in to an environment where the user may then enter the appropriate information after their credentials are verified. In this way, the time spent with the clinician may be optimized.

To address privacy concerns, a patient may be provided with a custom URL or QR code. A staff member may enter the URL into a browser on a tablet and provide the tablet to the patient. Once the patient has filled out the questionnaire, they may provide the tablet back to the clinician, and the clinician may click or tap on an icon such as a logo to return to a clean landing page for a future patient to access. Alternatively, the URL or QR code may be provided directly to the patient, such as through email or text message, and the patient may open the URL and enter their information. In this way, the risk of compromising patient data may be limited. Moreover, as noted herein, the patient's information may be accessible via a secure portal that is accessible when the user and/or clinician provides appropriate credentials.

After a patient fills out the questionnaire, they may be presented with another module, such as a communications or education module 160, which may provide the patient with information related to medical testing so that the patient is more informed and prepared prior to meeting with a medical professional. Such a module may include mobile-friendly interactive videos, text message reminders, and progress status updates. The videos may explain possible causes of the medical ailment, what genetic mutations are, risks and implications associated with the medical ailment, genetic testing and results, and genetic counseling preparation, among other such options. The videos may be dynamically provided to a patient based on their overall assessed risk, type of potential ailment, progress status, and date of upcoming appointment, among other options. The patient may also be provided with additional resources 150, including literature and other content related to the potential ailment and benefits of genetic counseling. According to another embodiment, a video chat bot may be provided if a patient has any questions or would like further information about the referral to seek genetic counseling.

A medical provider or clinician may have access to a dashboard which may provide information about a total number of patients who have been screened, a total number of high risk and low risk patients, a total number of appointments scheduled, and information associated with the education module. The medical provider or clinician may be able to see individual responses from a patient. In some embodiments, the individual responses may be aggregated and presented in a form of a summary report that the clinician can import into their own notes. The clinician may also be able to review progress statuses, such as whether the patient has scheduled a follow-up appointment or whether the patient has reviewed any educational materials related to genetic testing. Furthermore, in at least one embodiment, the dashboard may group patients into different categories (e.g., high risk, medium risk, low risk, etc.) and provide alerts to send follow up requests or reminders to patients based, at least in part, on their grouping. For example, a medium risk patient may be asked additional questions after a set period of time (e.g., approximately six months) to see if additional information may modify their grouping. Similarly, low risk patient may also receive a reminder, but over a longer period of time (e.g., approximately twelve months) to verify or otherwise check on the grouping. In this manner, care may be continued over periods of time as monitored by a clinician.

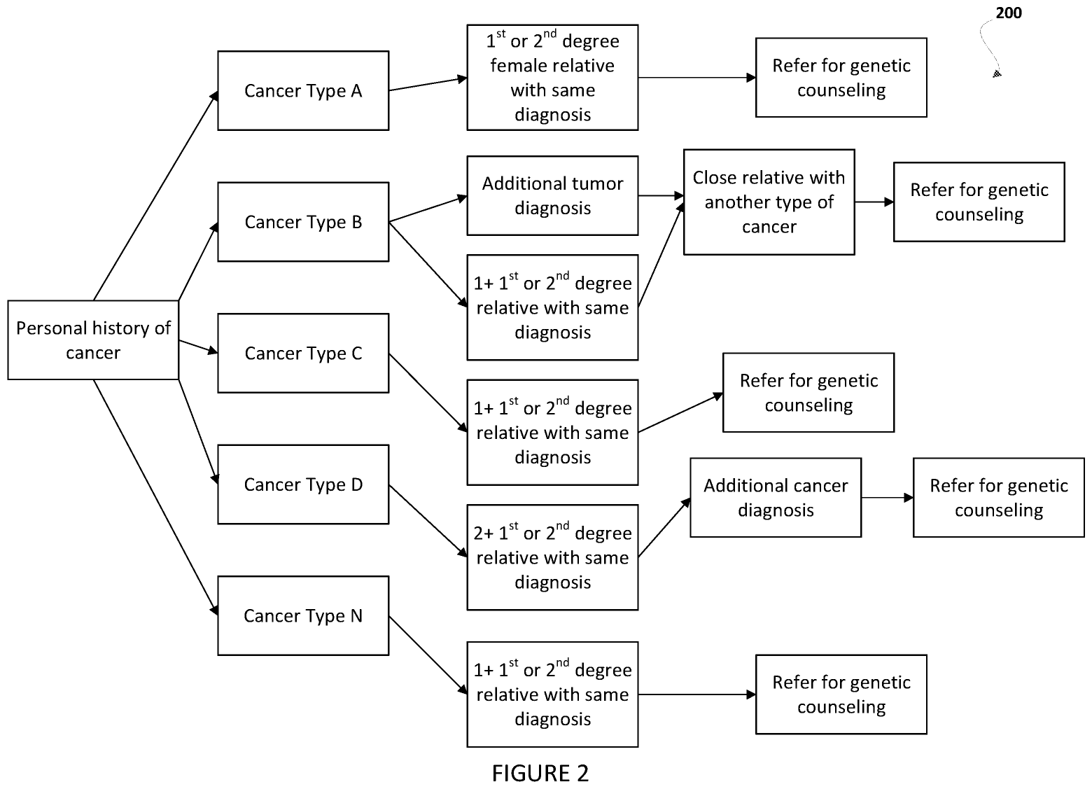

FIG. 2 illustrates an example decision tree data structure 200 that can be used in accordance with the various embodiments. While this example decision tree only shows some criteria, this example is not intended to be limiting and may include other branches and nodes depending on the type of cancer. Furthermore, other outcomes for the decision tree may be present, such as outcomes in which recommendations for genetic counseling are not recommended, recommendations for monitor one or more symptoms for a period of time, and/or the like. As explained, a user may be provided with a questionnaire including information that a specialist or counselor may ask, in addition to questions based on nationally-recognized guidelines and other questions which may be pertinent to early detection of a medical issue. Less pertinent or repetitive questions may be filtered out, leaving behind a specific subset of questions which may improve the likelihood of an early but accurate detection of a patient that would likely qualify for genetic testing. The questions being presented may be modified over time, for example, based on previously received answers. For example, a user may provide a first answer that filters out a portion of a set of second questions. However, a later answer may cause one or more of those previously filtered out questions to be more relevant, and those questions may then be included within a third set of questions, and so forth. In this manner, the system may adapt or otherwise adjust to user responses, thereby tuning the set of questions based on the input answers to provide a more personalized experience while also optimizing for time, relevancy, and/or confidence in recommendations. The overall time required for a user to fill out the questionnaire may be reduced to only a few minutes, instead of several minutes to an hour or more, due to the structured nature of the questionnaire. As previously mentioned, a user may spend significant amounts of time if they were to answer every single question in nationally-recognized guidelines or if the user were to see a specialist or counselor for early detection of a medical issue.

A list of risk factors or criteria identified for a type of medical condition may be provided to a user. For example, one or more risk factors or criteria for a specific type of cancer may be determined. Of those risk factors, a subset of risk factors determined to be most pertinent to the medical condition may be selected and/or identified. The subset of risk factors may be presented to a patient or user, such as in the form of a questionnaire. The subset of risk factors may include those factors which may be commonly attributed to an underdiagnosis of the targeted condition. In an example, risk factors may correspond to one or more criteria, such as “personal history of prostate cancer at any age” and “close relative with ‘X’ cancer”, where “X” denotes a specific type of cancer. A “close relative” according to this example may refer to a relative on the same side of the family who is a first, second, or third degree relative of the patient. A combination of criteria may be considered when determining whether to refer a patient to genetic counseling, such as age and history of cancer in combination with a known family history of cancer. In another embodiment, only a single criterion may need to be met in order to determine with sufficient confidence that the patient should be referred for genetic counseling. For example, a user having a known familial mutation or a relative with a known familial mutation regardless of personal or family history of cancer may always be referred for genetic counseling. The use of these criteria may improve the accuracy of a clinical recommendation, and may enable a user to seek genetic counseling earlier than they otherwise would have. Given the convenience of being able to take a shortened questionnaire at home, a user does not have to waste valuable time visiting a physical site to determine whether to seek genetic counseling.

In some cases, a given risk factor or criterion may require specific sub-decisions, such as “a patient's personal history of (a specified type of) cancer does not meet this criterion” when the questionnaire pertains to a different type of cancer. Other sub-decisions may include “must be separate close relatives with cancer on the same side of the family, not one relative with multiple cancer diagnoses,” among other such relationship mappings. Based on responses to the sub-decisions, one or more follow-up questions can be filtered out or otherwise eliminated to minimize the total amount of time a patient needs to complete the assessment or questionnaire. The criteria may be associated with a frequency score indicating how frequent a particular combination of criteria arises in a clinical visit. For example, patients may frequently be referred for genetic counseling if they have both a personal history of prostate cancer and the prostate cancer is metastatic. This information may be updated as new patient data is collected, and the information may be fed into a machine learning-based training system as training data to improve the prediction of potential risk for patients. Decisions and sub-decisions can be made based on various factors using implements including, but not limited to, algorithms, machine learning, and other such implements.

A classifier may be utilized to determine whether a patient is considered to be high-risk. The classifier may be a machine learning-based classifier. For each question answered with an affirmative answer and/or with an answer that includes an associated weight or score, a score may be generated. The scores for the questions may be aggregated and compared to one or more threshold values. The classifier may determine which recommendation to provide to the user depending on how the aggregated score compares to the threshold values. For example, if the aggregated score exceeds a threshold value, a recommendation to get tested for a genetic condition may be provided to a user. Additionally, a recommendation to seek counseling for a medical condition may be provided. Such a decision can be made using machine learning or other algorithms, to help provide a better understanding as to why the recommendation was made instead of merely providing data for presentation to a user. In an example, an indication about whether a user is high risk may be provided. If a score falls between two threshold values, an indication about a user being at risk, but not necessarily at high risk, may be provided. If a score falls below a determined threshold value, an indication that the user is low-risk or at no risk may be provided.

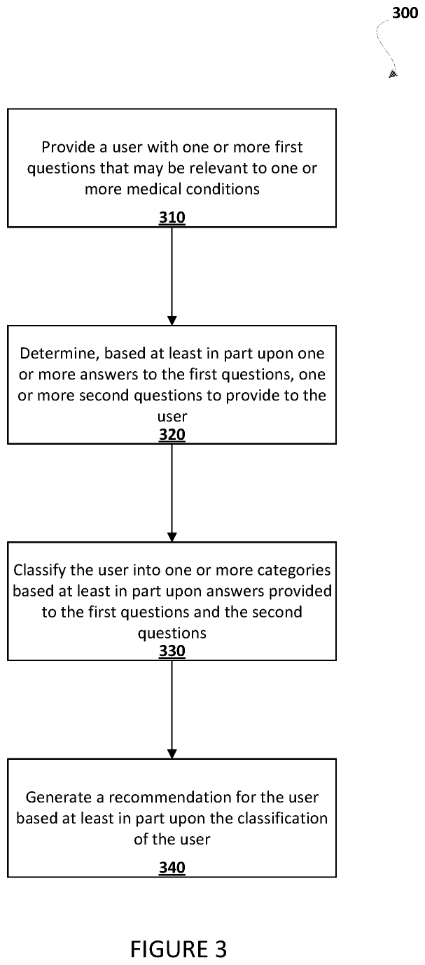

FIG. 3 illustrates an example method 300 that can be used in accordance with the various embodiments. It should be understood that for any process herein there can be additional, fewer, or alternative steps performed in similar or alternative orders, or in parallel, within the scope of the various embodiments unless otherwise specifically stated. In accordance with an example embodiment, a user can be provided with one or more first questions that may be relevant to one or more medical questions 310. Based at least in part upon one or more answers to the first questions, one or more second questions can be determined and provided to the user 320. The user can be classified into one or more categories based at least in part upon answers provided to the first questions and the second questions 330. A recommendation for the user can be generated based at least in part upon the classification of the user 340.

Various embodiments of the present disclosure may be used to provide a recommendation for a user and/or to group the user within one or more categories. The recommendation and/or groupings may be based, at least in part, on a subset of answers the user provides responsive to a set of questions. The subset of questions may change or otherwise be modified responsive to the answers provided by the user. For example, if the user provides a first answer to a first question, a first subset of second questions may follow. However, if the user provides a second answer, different from the first answer, a second subset of second questions may follow. In this manner, the user will not be provided with irrelevant questions, which could be time consuming and frustrating to answer. Accordingly, the user may be more likely to participate in and also complete the questions to receive a recommendation and/or grouping.

In at least one embodiment, questions may be presented to the user in the form of a limited set of responses that correspond to an input. For example, a first question may include a list of potential answers and a user input may correspond to selection of one or more of the potential answers, as shown in FIG. 1C. Alternatively, or in addition, the user may provide an open-form answer, which may then be evaluated using one or more machine learning systems, such as a large language model, to extract salient information for processing. By way of example, a user may be asked to summarize a history of cancer in their family, and the user may respond that “My dad had lung cancer.” The large language model may receive this input and identify salient features, such as “dad” and “lung cancer” to provide a mapping showing that a direct family member had a relevant cancer. Such a determination may then trigger further questions. Providing an open-ended set of questions may yield more rich data than only giving the user a subset of answers because the user may have information that is not provided as a potential answer and may, inadvertently, disregard that information as being unimportant or irrelevant if an option corresponding to that information is not present. Various embodiments may also mix potential answers such that the user can select from a set list and/or provide an open ended response, thereby providing multiple input paths for information.

As noted herein, one or more systems may classify or otherwise group a user based, at least in part, on their responses to a set or subset of questions. The classifications and/or groupings may be associated with one or more recommendations or follow-up prompts. For example, a user may receive a recommendation to receive genetic testing due to a classification that they are at a threshold risk of cancer based, at least in part, on answers to one or more question prompts. While the user may select whether or not to follow that recommendation, such a classification may also be used by a clinician, upon receiving consent from the user, to provide follow-up questions or recommendations. For example, the user may choose to delay genetic testing to see if any symptoms appear or due to various commitments in their life. The clinician may receive information that the user has not followed their recommendation, for example through a dashboard, and provide reminders or additional information over periods of time. In this manner, follow up care may be provided even if initial recommendations are not followed.

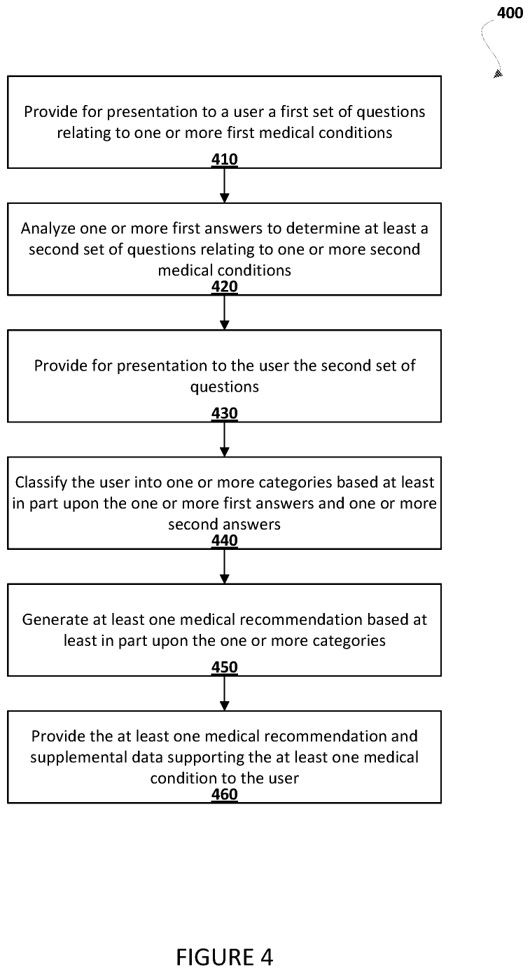

FIG. 4 illustrates an example method 400 that can be used in accordance with the various embodiments. It should be understood that for any process herein there can be additional, fewer, or alternative steps performed in similar or alternative orders, or in parallel, within the scope of the various embodiments unless otherwise specifically stated. In accordance with one or more embodiments, a first set of questions relating to one or more first medical conditions can be provided for presentation to a user 410. One or more first answers, received in response to the first set of questions, can be analyzed to determine at least a second set of questions relating to one or more second medical conditions, the second medical conditions including at least a subset of the one or more first medical conditions 420. The second set of questions can be provided for presentation to the user 430. The user can be classified into one or more categories based at least in part upon the one or more first answers and one or more second answers to the second set of questions 440. At least one medical recommendation can be generated Based at least in part upon the one or more categories 450. A medical recommendation can be provided for presentation to the user along with supplemental data supporting the at least one medical recommendation, where the supplemental data is determined based at least in part upon at least one of the one or more first answers, the one or more second answers, and the one or more categories 460.

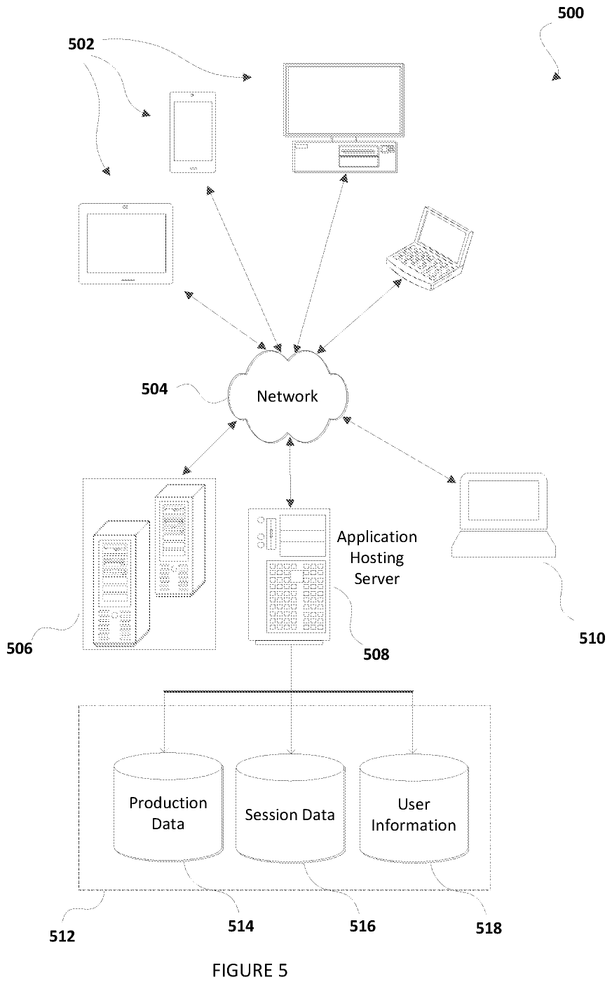

All or any portion of the above described can be implemented on a computing environment such as that illustrated in FIGS. 5-8. FIG. 5 shows an example of an environment 500 for implementing aspects in accordance with various embodiments. As will be appreciated, although a Web-based environment is used for purposes of explanation, different environments may be used, as appropriate, to implement various embodiments. The system includes an electronic client device 502, 510, which can include any appropriate device operable to send and receive requests, questions, messages or other information over an appropriate network 504 and convey information back to a user of the device. Examples of such client devices include personal computers, cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes a Web server 506 for receiving requests and serving content in response thereto, although for other networks, an alternative device serving a similar purpose could be used, as would be apparent to one of ordinary skill in the art.

The illustrative environment includes at least one application server 508 and a data store 512. It should be understood that there can be several application servers, layers or other elements, processes or components, which may be chained or otherwise configured, which can interact to perform tasks such as obtaining data from an appropriate data store. As used herein, the term data “store” refers to any device or combination of devices capable of storing, accessing and retrieving data, which may include any combination and number of data servers, databases, data storage devices and data storage media, in any standard, distributed or clustered environment. The application server 508 can include any appropriate hardware and software for integrating with the data store 512 as needed to execute aspects of one or more applications for the client device and handling a majority of the data access and business logic for an application. The application server provides access control services in cooperation with the data store and is able to generate content such as text, graphics, audio and/or video to be transferred to the user, which may be served to the user by the Web server 506 in the form of HTML, XML or another appropriate structured language in this example. The handling of all requests and responses, as well as the delivery of content between the client device 502, 510 and the application server 508, can be handled by the Web server 506. It should be understood that the Web and application servers are not required and are merely example components, as structured code discussed herein can be executed on any appropriate device or host machine as discussed elsewhere herein.

The data store 512 can include several separate data tables, databases or other data storage mechanisms and media for storing data relating to a particular aspect. For example, the data store illustrated includes mechanisms for storing production data 514 and user information 518, which can be used to serve content for the production side. The data store is also shown to include a mechanism for storing log or session data 516. It should be understood that there can be many other aspects that may need to be stored in the data store, such as page image information and access rights information, which can be stored in any of the above listed mechanisms as appropriate or in additional mechanisms in the data store 512. The data store 512 is operable, through logic associated therewith, to receive instructions from the application server 508 and obtain, update or otherwise process data in response thereto. In one example, a user might submit a search request for a certain type of item. In this case, the data store might access the user information to verify the identity of the user and can access the catalog detail information to obtain information about items of that type. The information can then be returned to the user, such as in a results listing on a Web page that the user is able to view via a browser on the user device 502, 510. Information for a particular item of interest can be viewed in a dedicated page or window of the browser. Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 5. Thus, the depiction of the system 500 in FIG. 5 should be taken as being illustrative in nature and not limiting to the scope of the disclosure. The various embodiments can be further implemented in a wide variety of operating environments, which in some cases can include one or more user computers or computing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially available operating systems and other known applications for purposes such as development and database management. These devices can also include other electronic devices, such as dummy terminals, thin-clients, gaming systems and other devices capable of communicating via a network.

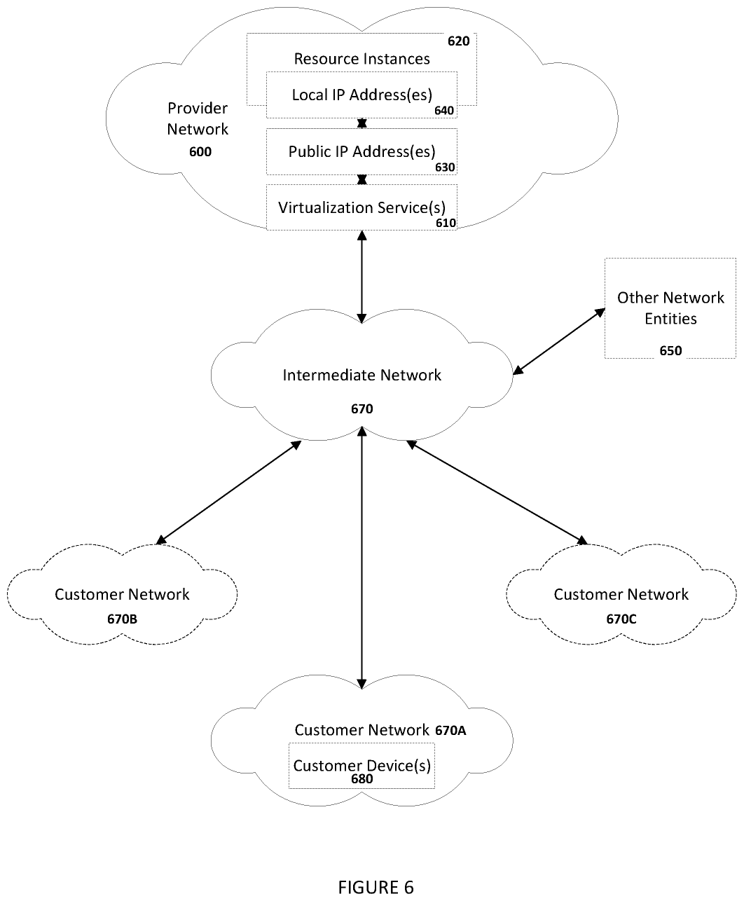

FIG. 6 illustrates an example provider network (or “service provider system”) environment according to some embodiments. A provider network 600 may provide resource virtualization to customers via one or more virtualization services 610 that allow customers to purchase, rent, or otherwise obtain instances 620 of virtualized resources, including but not limited to computation and storage resources, implemented on devices within the provider network or networks in one or more data centers. Local Internet Protocol (IP) addresses 640 may be associated with the resource instances 620; the local IP addresses are the internal network addresses of the resource instances 620 on the provider network 600. In some embodiments, the provider network 600 may also provide public IP addresses 630 and/or public IP address ranges (e.g., Internet Protocol version 4 (IPv4) or Internet Protocol version 6 (IPv6) addresses) that customers may obtain from the provider 600.

Conventionally, the provider network 600, via the virtualization services 610, may allow a customer of the service provider (e.g., a customer that operates one or more client networks 670A-670C including one or more customer device(s) 680) to dynamically associate at least some public IP addresses 630 assigned or allocated to the customer with particular resource instances 620 assigned to the customer. The provider network 600 may also allow the customer to remap a public IP address 630, previously mapped to one virtualized computing resource instance 620 allocated to the customer, to another virtualized computing resource instance 620 that is also allocated to the customer. Using the virtualized computing resource instances 620 and public IP addresses 630 provided by the service provider, a customer of the service provider such as the operator of customer network(s) 670A-670C may, for example, implement customer-specific applications and present the customer's applications on an intermediate network 660, such as the Internet. Other network entities 650 on the intermediate network 660 may then generate traffic to a destination public IP address 630 published by the customer network(s) 670A-670C; the traffic is routed to the service provider data center, and at the data center is routed, via a network substrate, to the local IP address 640 of the virtualized computing resource instance 620 currently mapped to the destination public IP address 630. Similarly, response traffic from the virtualized computing resource instance 620 may be routed via the network substrate back onto the intermediate network 660 to the source entity 650.

Local IP addresses, as used herein, refer to the internal or “private” network addresses, for example, of resource instances in a provider network. Local IP addresses can be within address blocks reserved by Internet Engineering Task Force (IETF) Request for Comments (RFC) 1918 and/or of an address format specified by IETF RFC 4193 and may be mutable within the provider network. Network traffic originating outside the provider network is not directly routed to local IP addresses; instead, the traffic uses public IP addresses that are mapped to the local IP addresses of the resource instances. The provider network may include networking devices or appliances that provide network address translation (NAT) or similar functionality to perform the mapping from public IP addresses to local IP addresses and vice versa.

Public IP addresses are Internet mutable network addresses that are assigned to resource instances, either by the service provider or by the customer. Traffic routed to a public IP address is translated, for example via 1:1 NAT, and forwarded to the respective local IP address of a resource instance.

Some public IP addresses may be assigned by the provider network infrastructure to particular resource instances; these public IP addresses may be referred to as standard public IP addresses, or simply standard IP addresses. In some embodiments, the mapping of a standard IP address to a local IP address of a resource instance is the default launch configuration for all resource instance types.

At least some public IP addresses may be allocated to or obtained by customers of the provider network 600; a customer may then assign their allocated public IP addresses to particular resource instances allocated to the customer. These public IP addresses may be referred to as customer public IP addresses, or simply customer IP addresses. Instead of being assigned by the provider network 600 to resource instances as in the case of standard IP addresses, customer IP addresses may be assigned to resource instances by the customers, for example via an API provided by the service provider. Unlike standard IP addresses, customer IP addresses are allocated to customer accounts and can be remapped to other resource instances by the respective customers as necessary or desired. A customer IP address is associated with a customer's account, not a particular resource instance, and the customer controls that IP address until the customer chooses to release it. Unlike conventional static IP addresses, customer IP addresses allow the customer to mask resource instance or availability zone failures by remapping the customer's public IP addresses to any resource instance associated with the customer's account. The customer IP addresses, for example, enable a customer to engineer around problems with the customer's resource instances or software by remapping customer IP addresses to replacement resource instances.

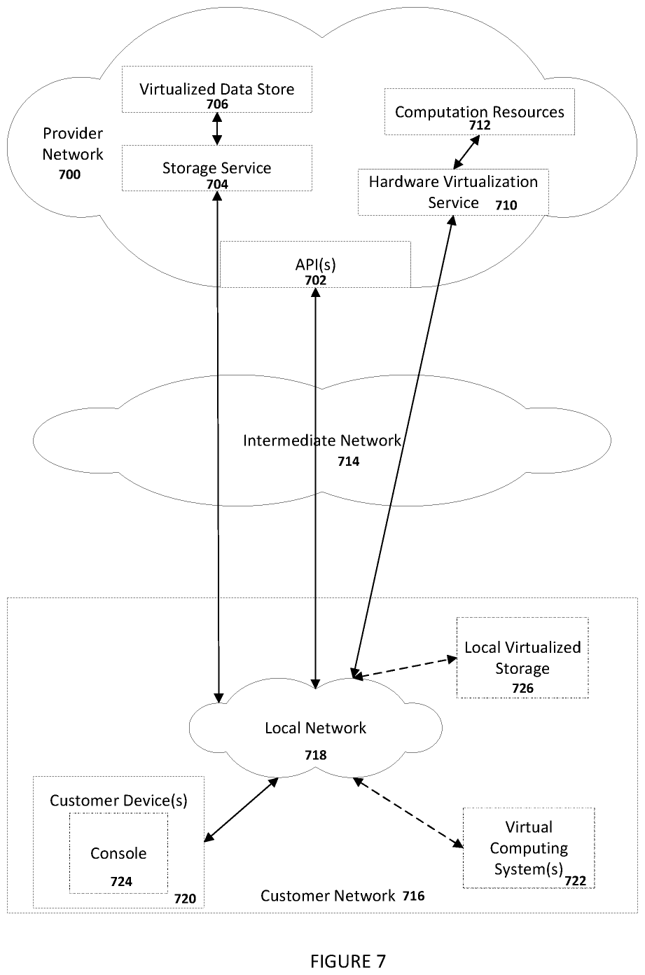

FIG. 7 is a block diagram of an example provider network that provides a storage service and a hardware virtualization service to customers, according to some embodiments. Hardware virtualization service 710 provides multiple computation resources 712 (e.g., VMs) to customers. The computation resources 712 may, for example, be rented or leased to customers of the provider network 700 (e.g., to a customer that implements customer network 716). Each computation resource 712 may be provided with one or more local IP addresses. Provider network 700 may be configured to route packets from the local IP addresses of the computation resources 712 to public Internet destinations, and from public Internet sources to the local IP addresses of computation resources 712.

Provider network 700 may provide a customer network 716, for example coupled to intermediate network 714 via local network 718, the ability to implement virtual computing systems 722 via hardware virtualization service 710 coupled to intermediate network 714 and to provider network 700. In some embodiments, hardware virtualization service 710 may provide one or more APIs 702, for example a web services interface, via which a customer network 716 may access functionality provided by the hardware virtualization service 710, for example via a console 724 (e.g., a web-based application, standalone application, mobile application, etc.). In some embodiments, at the provider network 700, each virtual computing system 722 at customer network 716 may correspond to a computation resource 712 that is leased, rented, or otherwise provided to customer network 716.

From an instance of a virtual computing system 722 and/or another customer device 720 (e.g., via console 724), the customer may access the functionality of storage service 704, for example via one or more APIs 702, to access data from and store data to storage resources of a virtual data store 706 (e.g., a folder or “bucket”, a virtualized volume, a database, etc.) provided by the provider network 700. In some embodiments, a virtualized data store gateway (not shown) may be provided at the customer network 716 that may locally cache at least some data, for example frequently-accessed or critical data, and that may communicate with storage service 704 via one or more communications channels to upload new or modified data from a local cache so that the primary store of data (virtualized data store 706) is maintained. In some embodiments, a user, via a virtual computing system 722 and/or on another customer device 720, may mount and access virtual data store 706 volumes via storage service 704 acting as a storage virtualization service, and these volumes may appear to the user as local (virtualized) storage 726.

While not shown in FIG. 7, the virtualization service(s) may also be accessed from resource instances within the provider network 700 via API(s) 702. For example, a customer, appliance service provider, or other entity may access a virtualization service from within a respective virtual network on the provider network 700 via an API 702 to request allocation of one or more resource instances within the virtual network or within another virtual network.

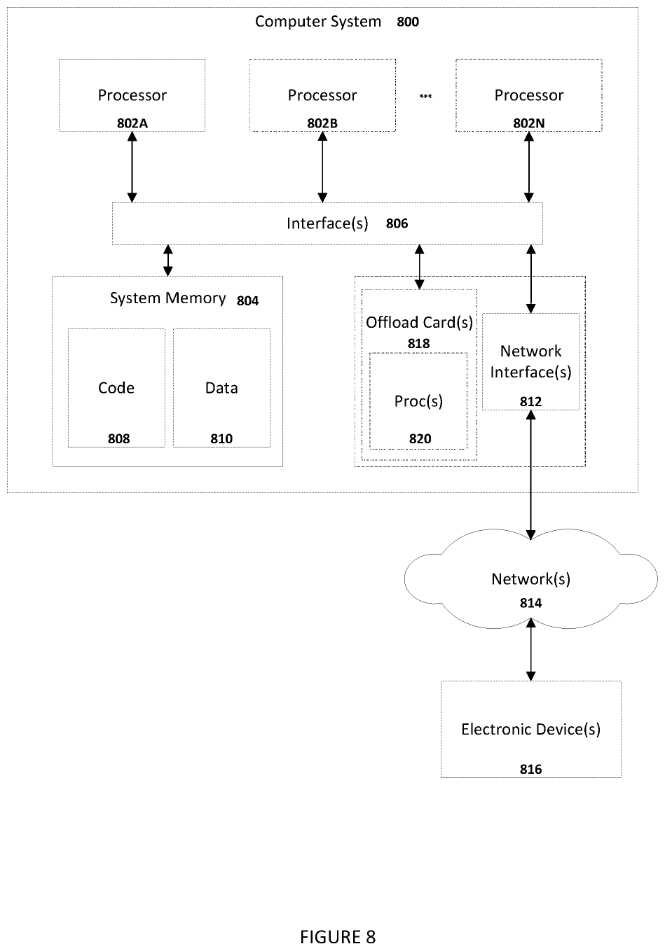

In some embodiments, a system that implements a portion or all of the techniques described herein may include a general-purpose computer system that includes or is configured to access one or more computer-accessible media, such as computer system 800 illustrated in FIG. 8. In the illustrated embodiment, computer system 800 includes one or more processors 802A-802N coupled to a system memory 804 via an input/output (I/O) interface 806. Computer system 800 further includes a network interface 812 coupled to I/O interface 806. While FIG. 8 shows computer system 800 as a single computing device, in various embodiments a computer system 800 may include one computing device or any number of computing devices configured to work together as a single computer system 800.

In various embodiments, computer system 800 may be a uniprocessor system including one processor 802A, or a multiprocessor system including several processors 802A-802N (e.g., two, four, eight, or another suitable number). Processors 802A-802N may be any suitable processors capable of executing instructions. For example, in various embodiments, processors 802A-802N may be general-purpose or embedded processors implementing any of a variety of instruction set architectures (ISAs), such as the x86, ARM, PowerPC, SPARC, or MIPS ISAs, or any other suitable ISA. In multiprocessor systems, each of processors 802A-802N may commonly, but not necessarily, implement the same ISA.

System memory 804 may store instructions and data accessible by processor(s) 802A-802N. In various embodiments, system memory 804 may be implemented using any suitable memory technology, such as random-access memory (RAM), static RAM (SRAM), synchronous dynamic RAM (SDRAM), nonvolatile/Flash-type memory, or any other type of memory. In the illustrated embodiment, program instructions and data implementing one or more desired functions, such as those methods, techniques, and data described above are shown stored within system memory 804 as code for one or more modules of the risk assessment system 808 and data 810.

In one embodiment, I/O interface 806 may be configured to coordinate I/O traffic between processor 802A, system memory 804, and any peripheral devices in the device, including network interface 812 or other peripheral interfaces. In some embodiments, I/O interface 806 may perform any necessary protocol, timing or other data transformations to convert data signals from one component (e.g., system memory 804) into a format suitable for use by another component (e.g., processor 802A). In some embodiments, I/O interface 806 may include support for devices attached through various types of peripheral buses, such as a variant of the Peripheral Component Interconnect (PCI) bus standard or the Universal Serial Bus (USB) standard, for example. In some embodiments, the function of I/O interface 806 may be split into two or more separate components, such as a north bridge and a south bridge, for example. Also, in some embodiments some or all of the functionality of I/O interface 806, such as an interface to system memory 804, may be incorporated directly into processor 802A.

Network interface 812 may be configured to allow data to be exchanged between computer system 800 and other devices 816 attached to a network or networks 814. In various embodiments, network interface 812 may support communication via any suitable wired or wireless general data networks, such as types of Ethernet network, for example. Additionally, network interface 812 may support communication via telecommunications/telephony networks such as analog voice networks or digital fiber communications networks, via storage area networks (SANs) such as Fibre Channel SANs, or via I/O any other suitable type of network and/or protocol.

In some embodiments, a computer system 800 includes one or more offload cards 818 (including one or more processors 820, and possibly including the one or more network interfaces 812) that are connected using an I/O interface 806 (e.g., a bus implementing a version of the Peripheral Component Interconnect Express (PCI-E) standard, or another interconnect such as a QuickPath interconnect (QPI) or UltraPath interconnect (UPI)). For example, in some embodiments the computer system 800 may act as a host electronic device (e.g., operating as part of a hardware virtualization service) that hosts compute instances, and the one or more offload cards 818 execute a virtualization manager that can manage compute instances that execute on the host electronic device. As an example, in some embodiments the offload card(s) 818 can perform compute instance management operations such as pausing and/or un-pausing compute instances, launching and/or terminating compute instances, performing memory transfer/copying operations, etc. These management operations may, in some embodiments, be performed by the offload card(s) 818 in coordination with a hypervisor (e.g., upon a request from a hypervisor) that is executed by the other processors 802A-802N of the computer system 800. However, in some embodiments the virtualization manager implemented by the offload card(s) 818 can accommodate requests from other entities (e.g., from compute instances themselves), and may not coordinate with (or service) any separate hypervisor.

In some embodiments, system memory 808 may be one embodiment of a computer-accessible medium configured to store program instructions and data as described above. However, in other embodiments, program instructions and/or data may be received, sent, or stored upon different types of computer-accessible media. Generally speaking, a computer-accessible medium may include non-transitory storage media or memory media such as magnetic or optical media, e.g., disk or DVD/CD coupled to computer system 800 via I/O interface 806. A non-transitory computer-accessible storage medium may also include any volatile or non-volatile media such as RAM (e.g., SDRAM, double data rate (DDR) SDRAM, SRAM, etc.), read only memory (ROM), etc., that may be included in some embodiments of computer system 800 as system memory 804 or another type of memory. Further, a computer-accessible medium may include transmission media or signals such as electrical, electromagnetic, or digital signals, conveyed via a communication medium such as a network and/or a wireless link, such as may be implemented via network interface 812.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof. In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C # or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc. Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information.

The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed. Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

What is claimed is:

1. A computer-implemented method, comprising:
providing, for presentation to a user, a first set of questions relating to one or more first medical conditions;
analyzing one or more first answers, received in response to the first set of questions, to determine at least a second set of questions relating to one or more second medical conditions, the second medical conditions including at least a subset of the one or more first medical conditions;
providing, for presentation to the user, the second set of questions;
classifying the user into one or more categories based at least in part upon the one or more first answers and one or more second answers to the second set of questions;
generating at least one medical recommendation based at least in part upon the one or more categories; and
providing, for presentation to the user, the at least one medical recommendation and supplemental data supporting the at least one medical recommendation, the supplemental data determined based at least in part upon at least one of the one or more first answers, the one or more second answers, and the one or more categories.

2. The computer-implemented method of claim 1, wherein the at least one medical recommendation is at least one of: a recommendation to schedule an appointment for genetic counseling, a recommendation to review materials related to genetic counseling, and a recommendation to explore educational content related to genetic counseling.

3. The computer-implemented method of claim 1, further comprising:
determining a respective score for individual responses to the one or more first questions and the one or more second questions;
classifying the user into the one or more categories based, at least in part, upon an aggregation of the respective scores for the individual responses; and
presenting a result of the classification to the user.

4. The computer-implemented method of claim 1, wherein the at least one medical recommendation is generated, at least in part, using a decision tree data structure.

5. The computer-implemented method of claim 1, further comprising:
assigning one or more weights to the one or more first answers and the one or more second answers.

6. The computer-implemented method of claim 1, wherein the user is classified into the one or more categories using a machine learning classifier.

7. A computer-implemented method, comprising:
obtaining medical history data for a person;
generating, based at least in part upon the medical history data, a first set of questions to be used to obtain a first set of information with respect to the person;
inferring, based at least in part upon at least a subset of a first set of answers to at least a subset of the first set of questions and the medical history data, at least a second set of information to be obtained with respect to the person; and
generating a second set of questions to be used to obtain the second set of information;
inferring one or more medical actions to be taken with respect to the person based, at least in part, upon the first set of information and the second set of information; and
providing a recommendation specifying the one or more medical actions and including supplemental information supportive of the recommendation, the supplemental information determined based at least in part upon the first set of information, the second set of information, and the medical history data.

8. The computer-implemented method of claim 7, wherein the recommendation is a recommendation to schedule an appointment for genetic counseling.

9. The computer-implemented method of claim 7, further comprising:
determining respective scores for individual answers to at least the subset of the first set of questions and at least a subset of the one or more second questions;
classifying the person into one or more categories based, at least in part, upon an aggregation of the respective scores for the individual answers; and
presenting a result of the classification to the person.

10. The computer-implemented method of claim 7, wherein the recommendation is generated, at least in part, using a decision tree data structure.

11. The computer-implemented method of claim 7, further comprising:
assigning one or more weights to the subset of the first set of answers and at least a subset of a second set of answers to at least a subset of the second set of questions.

12. The computer-implemented method of claim 7, wherein the person is classified into the one or more categories using a machine learning classifier.

13. The computer-implemented method of claim 7, further comprising:
classifying the person into one or more categories based at least in part upon the subset of the first set of answers and at least a subset of a second set of answers responsive to the second set of questions.

14. A non-transitory computer-readable medium comprising instructions which, when executed by at least one processor, cause the at least one processor to:
determine a set of questions determined to be relevant for a medical condition;
determine a subset of the questions for presentation to a user;
receive one or more responses to the subset of the questions from the user;
determine scores for individual responses of the one or more responses;
classify the user into one or more categories based, at least in part, upon an aggregation of the scores; and
present a result of the classification, pertinent to the medical condition, to the user.

15. The non-transitory computer-readable medium of claim 14, wherein the result of the classification is at least one of: a recommendation to schedule an appointment for genetic counseling, a recommendation to review materials related to genetic counseling, and a recommendation to explore educational content related to genetic counseling.

16. The non-transitory computer-readable medium of claim 14, wherein the instructions, when executed by the at least one processor, cause the at least one processor to further:
analyze the one or more responses to the subset of the questions to determine at least a second set of questions relating to one or more second medical conditions;
provide, for presentation to the user, the second set of questions;
classify the user into the one or more categories based at least in part upon the one or more responses to the subset of questions and one or more second responses to the second set of questions; and
present a second result of the classification to the user based at least in part upon the one or more responses to the subset of questions and the one or more second responses.

17. The non-transitory computer-readable medium of claim 14, wherein at least one of the result or the second result is generated, at least in part, using a decision tree data structure.

18. The non-transitory computer-readable medium of claim 14, wherein the instructions, when executed by the at least one processor, cause the at least one processor to further:
assign one or more weights to the one or more responses to the subset of the questions from the user.

19. The non-transitory computer-readable medium of claim 14, wherein the one or more categories are indicative of a risk of the user having the medical condition.

20. The non-transitory computer-readable medium of claim 14, wherein the user is classified into the one or more categories using a machine learning classifier.
